(19) United States
(12) Patent Application Publication
(10) Pub. No.: US 2010/0304458 A1
(43) Pub. Date: Dec. 2, 2010

(54) Hydrogen And Electrical Current Production From Photosynthetically Driven Semi Biological Devices (SBDs)

(75) Inventor: Paolo Bombelli, Cambridge (GB)

Correspondence Address: Pepper Hamilton LLP, 400 Berwyn Park, 899 Cassatt Road, Berwyn, PA, 19312-1183, US

(73) Assignee: H PLUS ENERGY LIMITED, Oxford (GB)

(21) Appl. No.: 12/680,327

(22) PCT Filed: Sep. 26, 2008
(86) PCT No.: PCT/GB08/03278

§ 371 Date: Jun. 23, 2010

(30) Foreign Application Priority Data
Sep. 28, 2007 (GB) 0719009.3

(51) Int. Cl. C12P 3/00; C12M 1/00; H01M 8/16

(52) U.S. Cl. 435/168; 435/292.1; 429/2

(57) ABSTRACT

The present invention provides a device comprising a first chamber and a second chamber, the first chamber oriented in two alternative ways (1) the first chamber having an anode in contact with an aqueous solution comprising a photosynthetic organism or photosynthetic part thereof and an electron acceptor molecule, an inlet and an outlet, OR (2) the first chamber having direct contact between an anode and the photosynthetic organism, the second chamber having a cathode in contact with an aqueous solution of an electrolyte, an outlet, wherein the anode and the cathode are connected by a switched electric circuit optionally having an external power source and wherein the second chamber is separated from the first chamber by a proton selective membrane. The device described in the present inventions allows for the production of hydrogen and electrical current.

The present invention relates to a device and method for the production of hydrogen or electrical current using a photosynthetic process.

Photosynthesis is the most valuable method to harness the energy of light; the primary products of this process (oxygen, protons and electrons) can be used to produce hydrogen or electrical current.

Hydrogen is viewed as one of the best potential energy carriers for the future; the gas can react with oxygen in a fuel cell generating an electrical current and leaving water as the only by-product. Fuel cell technology has led to hydrogen as being perceived as a clean, renewable source of energy. However, the current method of choice for the large-scale production of hydrogen is steam reformation of fossil fuels, which, like many other production methods, releases carbon dioxide as a by-product. Photosynthetic microorganisms, such as cyanobacteria and green algae represent attractive models for environmentally “clean” bio-hydrogen production, since they can be engineered to produce hydrogen from light through the activity of the photosynthetic apparatus. However, the hydrogenase enzyme—which is responsible for the production of hydrogen—is inhibited by oxygen. Here we show that photosynthetic organisms can be used to produce hydrogen in the presence of oxygen in a novel semi-biological device that physically separates photochemistry from hydrogen production.

Electrical current can be created employing the electrons generated during photosynthetic activity. However, the current methods of choice for production of electrical current require the use of fossil fuels, which, like many other production methods, release carbon dioxide as a by-product or thermonuclear reaction. Photosynthetic membranes, such as thylakoids extracted from photosynthetic organisms (green algae and plants) represent an attractive model for environmentally “clean” bio-electrical current production, since they can produce electrical current from light through the activity of their photosynthetic apparatus. However, their short life time outside a biological context—which is reason behind the easier accessibility to electrons—limits their large-scale use. Here we show that whole photosynthetic organisms can be used to produce electrical current instead of thylakoid membranes in a novel semi-biological device that physically interacts with the biological material.

Chlamydomonas reinhardtii, a unicellular eukaryotic green alga, has been used as a model organism to study a number of fundamental biological processes. Through the catalytic activity of photosystem II (PSII), C. reinhardtii is able to split water into oxygen, hydrogen ions and electrons; the electrons are funnelled through the photosynthetic chain to the hydrogenase enzyme, which combines two electrons and two protons, releasing hydrogen gas. Since the hydrogenase enzyme is inhibited by extremely low concentrations of oxygen, this process can occur under anaerobic conditions only. The current method to induce photofermentative hydrogen production in C. reinhardtii involves starving the organism of sulphur, which reduces the activity of the photosynthetic chain, such that there is no net production of oxygen and the cultures become anaerobic.

There is therefore a need to produce hydrogen by means of a process that is not harmful to the environment and which overcomes the problems associated with the culture of photosynthetic organisms where the presence of oxygen can inhibit production of hydrogen.

It has now been found that by constructing a transparent multi-chamber device that allows light to penetrate into at least one of the chambers, such problems can be successfully overcome with the concurrent production of hydrogen gas.

According to a first aspect of the invention, there is provided a device comprising a first chamber and a second chamber (of any size), The first chamber can be set in two different ways. 1) the first chamber having an anode in contact with an aqueous solution comprising a photosynthetic organism or photosynthetic part thereof and an electron acceptor molecule, an inlet and an outlet, or 2) the first chamber having a direct contact between the anode and the photosynthetic organism. In this last case the electron acceptor is no longer required and the electron transport, inlet and outlet, is mediated by transmembrane proteins. The second chamber having a cathode in contact with an aqueous solution of an electrolyte, an inlet and an outlet, where the anode and the cathode are connected by a switched electric circuit optionally having an external power source and wherein the second chamber is separated from the first chamber by a proton selective membrane. If the external power source is not provided, the cathodic reaction is driven by the formation of water. In this context the main product is the electrical current passing through the external circuit between anode and cathode. If the chambers are large, chamber 1 may comprise of an open algal pond. Alternatively, if the chambers are small (microfabricated) the two chambers may be on the μm scale. In such a microfabricated arrangement, the complete device may consist of multiple chambers, which may be electrically connected to form a panel.

Various different embodiments of this aspect of the invention are therefore possible. For example, the first and second chamber may be arranged such that the second chamber is contained within the first chamber. In such an embodiment, the entire second chamber will be separated from the first chamber by the proton selective membrane. However, in other embodiments, the first and second chambers may be constructed as adjacent chambers in which the connecting surface between the adjacent chambers is the proton selective membrane. Where the photosynthetic system donates electrons directly (mediator-less) to the anode, a physical barrier between the two chambers may not be necessary. The first chamber and the second chamber may therefore form a single chamber in this arrangement where no barrier is present.

The first chamber may be constructed of any suitable transparent material in order that it can be used to support the growth or culture or maintenance of a photosynthetic organism or a part thereof. For example, materials that generally have a smooth surface such as glass, concrete, Perspex™, plastic, metal (e.g. stainless steel) may be used. The second chamber may be composed of similar materials but at least a portion of the external surface will be composed of a proton selective material in order to permit ion flow between the lumen of the first chamber and the lumen of the second chamber.

The photosynthetic organism or part thereof may be a thylakoid or thylakoid membrane, plant or plant tissue, cyanobacteria (or other photosynthetic bacterium), eukaryotic algae. Suitably, a population of such organisms or photosynthetic parts thereof may be present in the first chamber of the device.

Thylakoids (which are also sometimes known as thylakoid membranes) are a phospholipid bilayer membrane-bound compartment contained inside a photosynthetic bacterium or a plant or algal cell chloroplast.

For thylakoid membrane preparation plant tissue may be used, such as terrestrial plants, e.g. spinach, lettuce, beet (e.g. Sugar beet), cereals (e.g. wheat, barley, maize), grass, or alternatively, aquatic plants e.g. Posidoniaceae, Zosteraceae, Zostera, Heterozostera, Phyllospadix, Enhalus, Halophila, Thalassia, Amphibolis, Cymodocea, Halodule, Syringodium, Thalassodendron. Plant tissue includes leaves, stems, calli, cells or parts thereof.

Cyanobacteria that might be used include Anabaena, Crocosphaeri, Phormidium, Gloeobacter (or any other cyanobacterium in which the photosynthetic electron transport chain is exposed to the periplasm or cell surface), Nostoc punctiforme, Nostoc sp., Prochlorococcus marinus, Synechococcus elongatus, Synechococcus sp, Thermosynechococcus elongatus, Trichodesmium erythraeum.

Eukaryotic algae may include Antithamnion, Ascophyllum, Atractophora, Audouinella, Botryococcus, Charales, Chlamydomonas, Chlorella, Chlorogonium, Chondrus, Cladophora, Codium, Coleochaete, Corallina, Cryptomonas, Cyanidioschyzon, Cyanidium, Dasya, Desmids, Dunaliella, Dysmorphococcus, Enteromorpha, Euglena, Falosphaera, Fucus, Haematococcus, Isochrysis, Laminaria, Lemanea, Mougeotia, Nannochloris, Nannochloropsis, Neochloris, Pelvetia, Phacotus, Phaeodactylum, Platymonas Pleurochrysis, Polytoma, Polytomella, Porphyridium, Prymnesium, Pyramimonas, Scenedesmus, Spirogyra, Spirulina, Spyridia, Tetraselmis, Tetraspora, Thalassiosira, Ulva, Volvox, Zygnema.

The electron acceptor molecule may be any electrochemically active compound capable of transferring electrons from the photosynthetic material to the anode. Many different organic and organometallic compounds could work in the device; these include, but are not limited to thionines (e.g. acrylamidomethylthionine, N,N-dimethyl-disulfonated thionine etc), viologens (e.g. benzylviologen, methylviologen, polymeric viologens etc), quinones (e.g. 2-hydroxy-1,4-naphthoquinone, 2-methyl-1,4-naphthoquinone, 2-Methylnaphthoquinone etc), phenazines (e.g. phenazine ethosulfate, safranine, etc), phenothiazines (e.g. alizarine brilliant blue, methylene blue, phenothiazine, toluidine blue, etc), phenoxazines (e.g. brilliant cresyl blue, gallocyanine, resorufin, etc), Iron cyanide, Ferric chelate complexes (e.g. Fe(III)EDTA), Ferrocene derivates, Iron cyanide, Dichlorophenolindophenol, Diaminodurene.

The anode may be composed of platinum, platinum-black, gold, silver, indium tin-oxide (ITO), carbon, reticulated vitreous carbon, carbon felt, glassy carbon, graphite, graphite felt, a noble metal, any solid or porous conductive plastic, or a mixture of any thereof.

The aqueous solution may be a buffered medium or a buffered growth medium to culture stabilizes the photosynthetic organism or part thereof. For example the medium may therefore buffer and/or culture the thylakoid membranes, or buffer and/or culture the photosynthetic organism to support growth. Examples of such aqueous growth media may contain a source of nitrogen such as ammonia, nitrate or urea, a source of phosphate, such as potassium phosphate, or sodium phosphate, a source of magnesium, such as magnesium sulphate, a source of calcium, such as calcium chloride, a number of essential trace elements or ions including, Iron, Zinc, Borate, Manganese, Cobalt, Copper, Molybdate and/or Silicate.

The first chamber comprises an inlet port, and an outlet port, to allow the aqueous solution to continuously flow through the device.

The first chamber may be constructed as a sealed chamber or as a partially open chamber, optionally provided with a removable covering. Such a removable covering would allow oxygen evolved from the chamber to be collected, it would prevent littering of the chamber from items found naturally in the environment, and it would also allow the temperature of the chamber to be regulated. If the chamber is constructed as a sealed chamber then a vent or pressure valve can be included.

The first chamber may be constructed to allow the direct contact between the anode and the photosynthetic organism. In this way a photosynthetic biofilm covering the anode surface is formed. The electron acceptor is therefore no longer required and the electron transport across the plasmamembrane of the photosynthetic organism, inlet and outlet, is mediated directly by transmembrane proteins (such as ferro reductase, Fe-chelate reductase, NADH oxidase and NADPH oxidase).

The second chamber may be as described above adjacent to the first chamber or contained within the first chamber. A proton selective membrane will separate the second chamber from the first chamber at least partially.

The cation exchange membrane that separates the second chamber from the first chamber may be a polytetrafluoroethylene membrane, for example a NAFION™ membrane.

NAFION™ is a perfluorinated polymer that contains small proportions of sulfonic or carboxylic ionic functional groups. Its chemical structure is attached below:

X=is either a sulfonic or carboxylic functional group
M=is either a metal cation in the neutralized form or an H+ in the acid form.

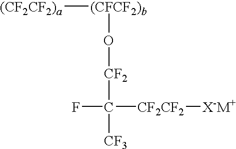

The electrolyte solution in the second chamber may be composed of an aqueous solution of a suitable salt, for example a halide salt of an alkali metal or an alkaline earth metal, for example potassium fluoride, chloride, bromide or iodide.

The cathode in the second chamber is where hydrogen will be produced. The cathode may be made from, but is not limited to, the following materials: platinum, palladium, metals (such as gold, steel or copper) coated with platinum, platinum coated with a hydrogenase enzyme.

The second chamber is also provided with an outlet through which hydrogen produced in the second chamber is released from the device.

The anode in chamber 1 will be connected to the cathode in chamber 2 by an external electrical circuit. This circuit may be composed of insulated electrical wiring (preferably made from copper), and a switch. The switch will allow electrical energy, derived from an external power source device (such as mains electricity, photovoltaic cell, wind farm etc) to be fed into the electrical circuit. The extra power allows electrons to flow from the anode in the first chamber to the cathode in the second chamber where they are consumed for hydrogen production.

If the external power source is not provided the cathodic reaction is driven by the formation of water. In this context the main product is the electrical current passing through the external circuit between anode and cathode. In this case the cathode may be made from, but is not limited to, the following materials: platinum, metals (such as gold, steel or copper) coated with platinum, other conductive material coated with laccase enzyme.

According to a second aspect of the invention, there is provided a method for the generation of hydrogen from a device according to the first aspect of the invention system, the method comprising the steps of
(1) operating the switch to connect the anode to the cathode, and
(2) introducing a source of additional bias potential (electrons) from an external power source.

According to a third aspect of the invention, there is provided a method for the generation of electrical current from a device according to the first aspect of the invention system, the method comprising the steps of
(1) operating the switch to connect the anode to the cathode, and
(2) introducing a spontaneous reaction as a driving force happening at the cathode surface (oxygen reduction into water).

Preferred features for the second and subsequent aspects of the invention are as for the first aspect mutatis mutandis.

The present invention will now be further described by way of illustration with reference to the accompanying drawings in which:

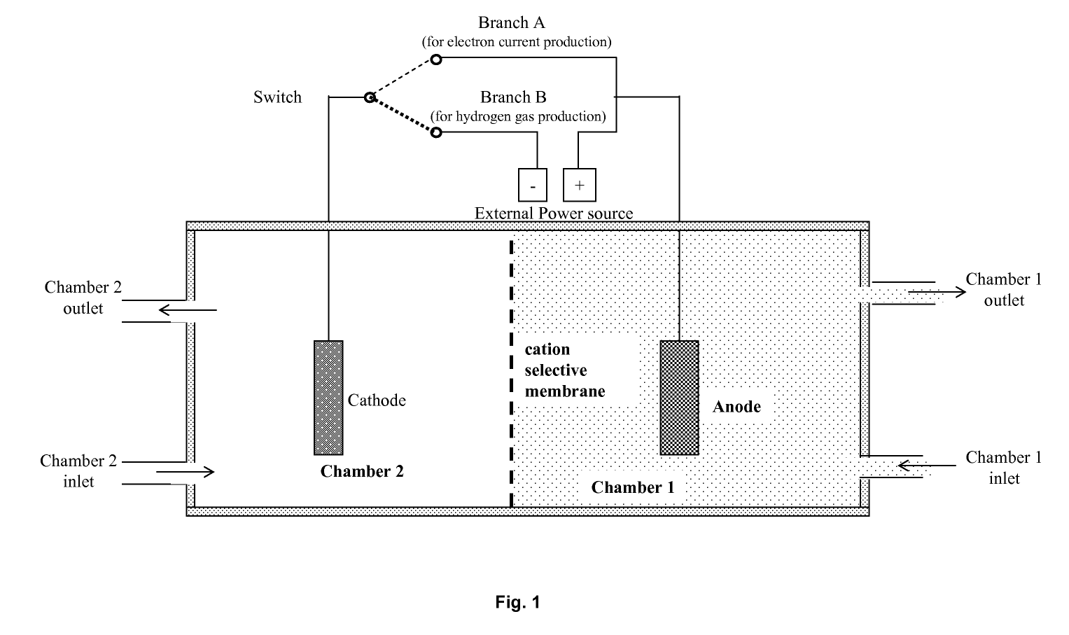

FIG. 1 shows a diagram representing a device of the invention for the production of hydrogen or electrical current.

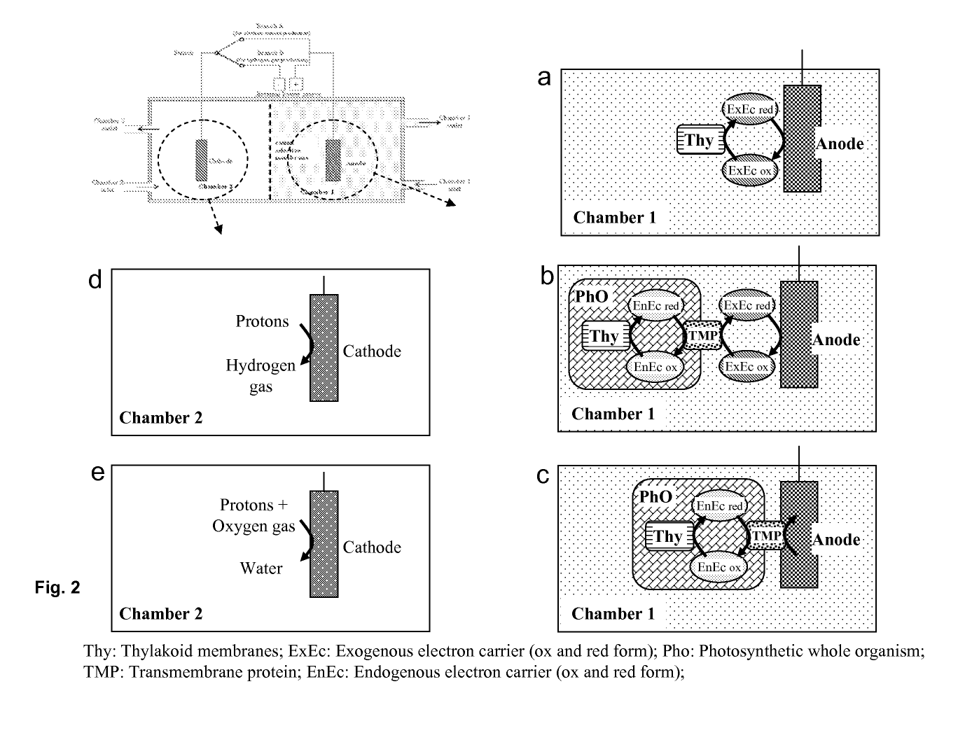

FIG. 2 shows three different ways of electron transport that can occur at anodic chamber and two alternative cathodic reaction.

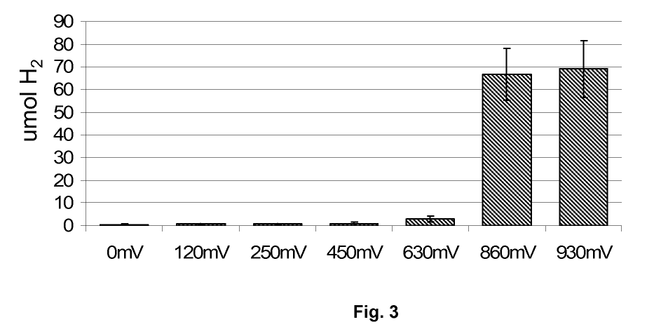

FIG. 3 shows the effect of increasing the amount of external energy supplied to the device on hydrogen production using thylakoid membrane as photosynthetic material.

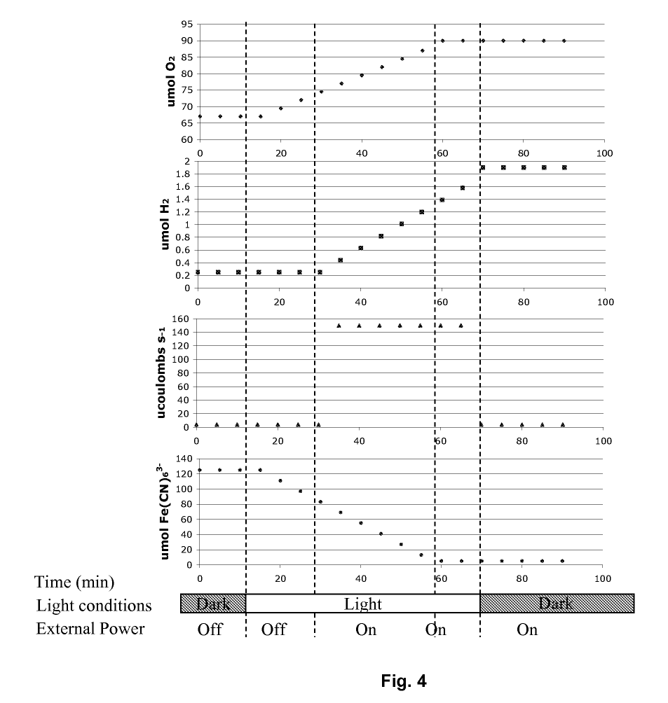

FIG. 4 shows the effect of light and the external power source on the device using thylakoid membrane as photosynthetic material.

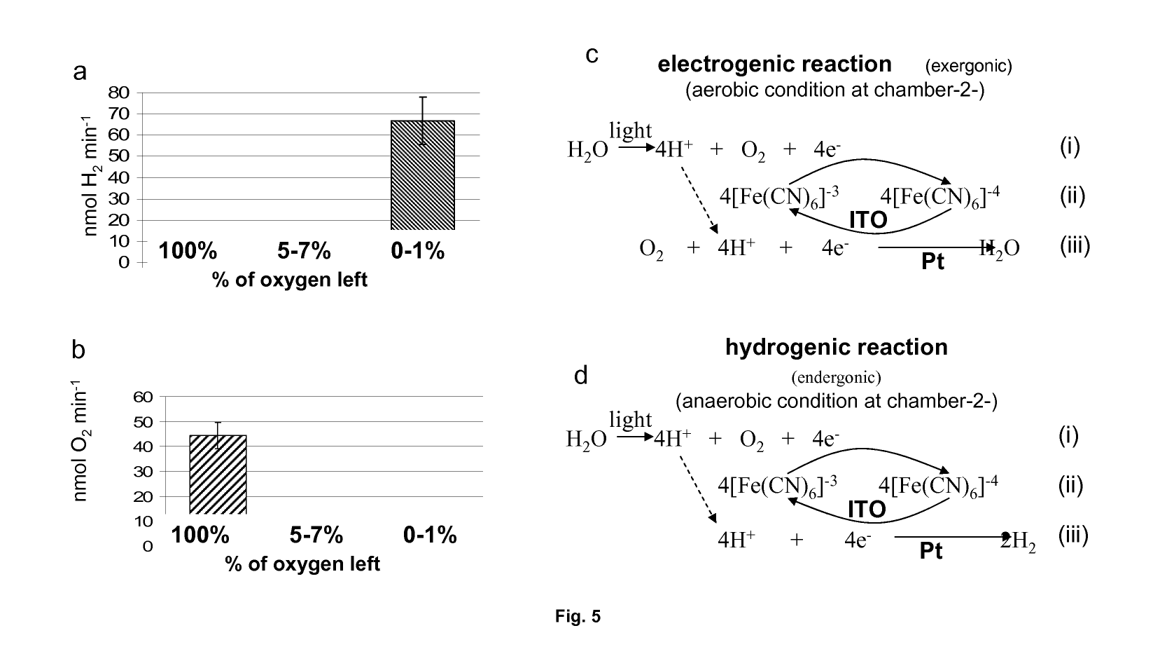

FIG. 5 shows the effect of oxygen in chamber 2 of the device using thylakoid membrane as photosynthetic material.

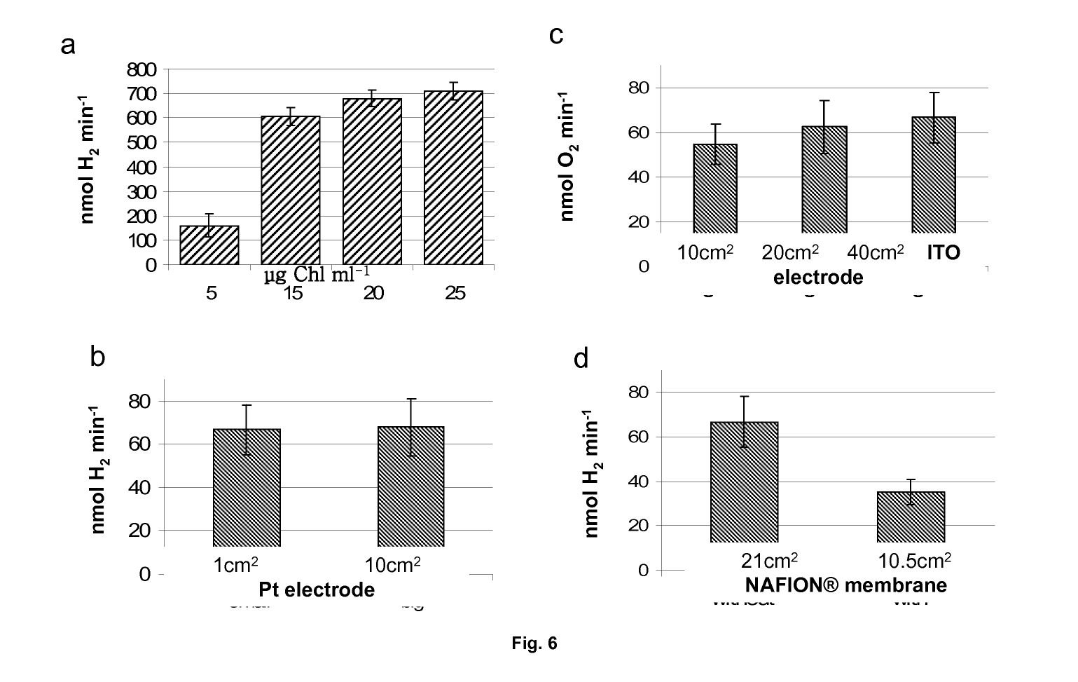

FIG. 6 shows the effect of individual components in the semi-biological device using thylakoid membrane as photosynthetic material

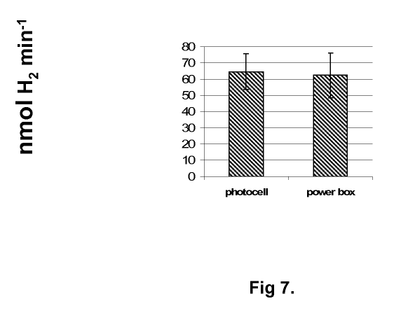

FIG. 7 shows the external energy can be supplied from different sources using thylakoid membrane as photosynthetic material.

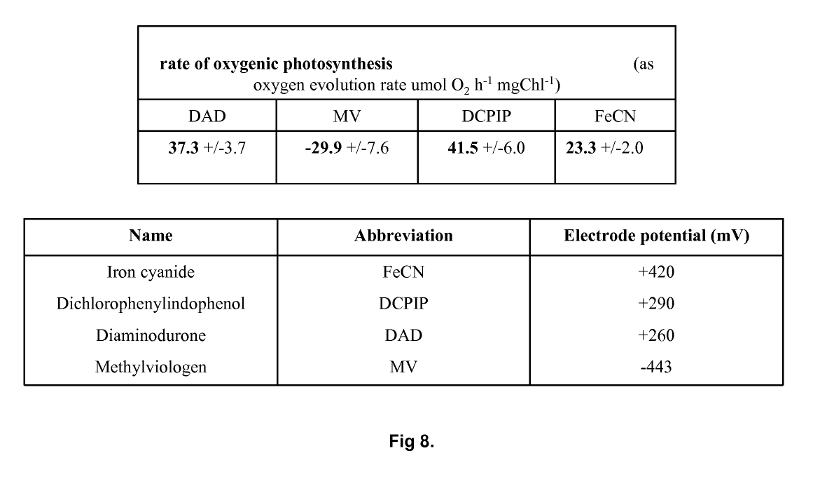

FIG. 8 shows the performance of the device when different electron carriers are used the aqueous solution in chamber 1 using thylakoid membrane as photosynthetic material.

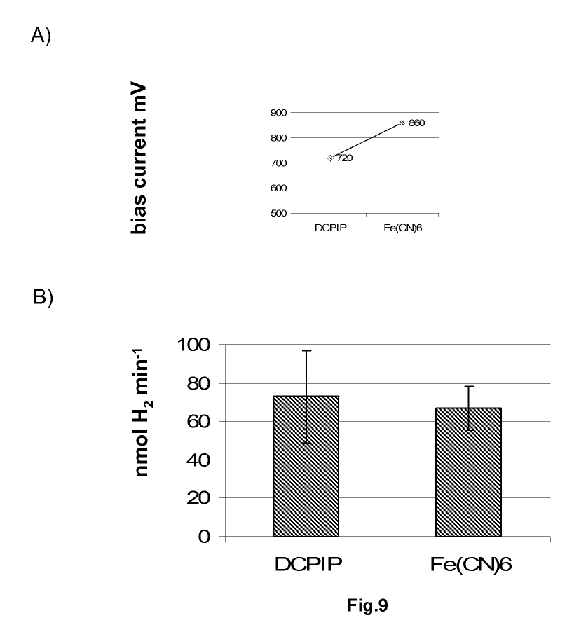

FIG. 9 shows a comparison between using Fe(CN)₆, Diaminodurene, metilviologen and dichlorophenylindophenol as the electron carriers in the aqueous solution in chamber 1 represented as photosynthetic oxygenic activity

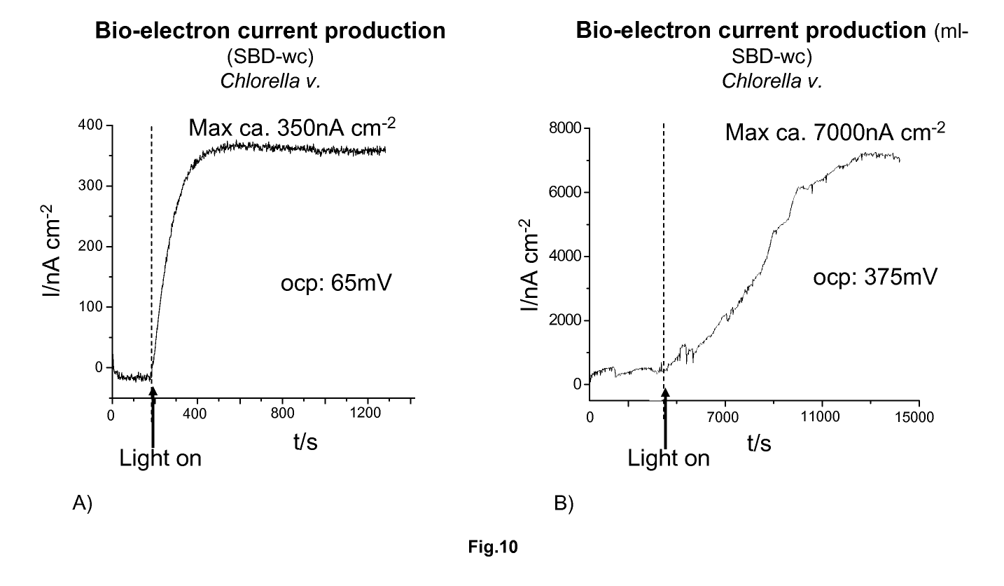

FIG. 10 shows the performance of the device when a whole photosynthetic organism is used as photosynthetic material.

Briefly, in FIG. 1, the device consists of two chambers. Chamber 1 and chamber 2 are side-by-side. Chamber 1 may be open to the environment, or sealed within a case, such as for example plastic, glass, or Perspex™. Chamber 1 contains photosynthetic material suspended in growth medium. There is a continuous flow of fresh medium, and new cells into chamber 1 through the inlet port, and a continuous flow of old cells, and spent medium out of the chamber through the outlet port. In addition to the photosynthetic material, chamber 1 also comprises an anode, and optionally an electrochemically active compound capable of transferring electrons from the photosynthetic material to the anode.

The contents of chamber 2 are separated from chamber 1 by a proton selective membrane (e.g. NAFION) that allows hydrogen ions to freely diffuse between the chambers, but prevents the diffusion of all of the other components. Chamber 2 also contains a cathode submerged in an aqueous solution of an electrolyte, for example a halide salt of an alkali earth metal, or of an alkaline earth metal, for example potassium chloride, an outlet port, which allows hydrogen gas to be removed from the chamber, and an inlet port which allows the chamber to be filled with electrolyte.

The cathode in chamber 2 and the anode in chamber 1 are connected to each other by electrical wiring to form a circuit. The circuit contains a switch that allows an additional source of energy to be fed into the circuit. When the circuit switch is turned on in branch B and sunlight simultaneously shines on chamber 1, the device is in operation reducing the electrochemically active compound in the chamber, which will then donate electrons to the anode. Electrons will flow to the cathode, where hydrogen will be produced. The flow of electrons from the anode to the cathode may be made thermodynamically favourable by the addition of extra energy from an external power source. The hydrogen produced at the cathode in chamber 2 will be removed from the system via the outlet port in chamber 2. When the circuit switch is turned on in branch A and sunlight simultaneously shines on chamber 1, the device is in operation reducing the electrochemically active compound, in the chamber, which will then donate electrons to the anode. Electrons will flow to the cathode, where water will be produced. The flow of electrons from the anode to the cathode is thermodynamically favourable by the exergonic property of water formation.

FIG. 1 shows a device of one embodiment of the invention with a reaction scheme for the production of hydrogen or electrical current. Thylakoid membranes (PS), placed in chamber 1, are used to reduce a soluble electron carrier, which in this case is Fe(CN)₆. The soluble electron carrier transfers electrons from the photosynthetic electron transport chain to an Indium Tin Oxide (ITO) covered glass slide, which acts as the anode. The electrons flow through a copper wire to a platinum cathode placed in chamber 2, which catalyses the production of hydrogen gas. Hydrogen ions are able to freely diffuse through a NAFION membrane between the chambers. A photovoltaic cell, placed behind chamber 1 supplies a bias potential (current), which makes the flow of electrons to the platinum cathode thermodynamically favourable and allows hydrogen production. A switch in the copper wire allows the bias potential (current) to be turned on or off. Under this condition electrical current is generated concurrently with the water production at the cathode surface. A hydrogen electrode in chamber 2 and oxygen electrodes in both chambers are able to monitor the production of the respective gases. A potentiostat monitors the amount of electrical current passing through on the external circuit.

FIG. 2 shows in detail how the electron chains occur in the two chambers (Chamber 1 is the anodic one and Chamber 2 is the cathodic one).

Chamber 1 contains photosynthetic material suspended in growth medium, an anode, and optionally an electrochemically active compound capable of bridging electron flow from the photosynthetic material to the anode. In panel 2a, b and c we describe three different ways to connect the electrode. In FIG. 2a, the thylakoid membranes (Thy) reduce a soluble exogenous electron carrier (ExEc), which in this case is Fe(CN)₆³⁻. This reducted red-ox shuttle transfers electrons to an anode, which in this case is Indium Tin Oxide (ITO). In FIG. 2b, an Photosynthetic whole organism (PhO) reduces a soluble exogenous electron carrier (ExEc), which in this case is Fe(CN)₆³⁻. This red-ox reaction occurs through the intermediate activity of endogenous electron carriers (EnEc) and a transmembrane protein or proteins (TMP). The electron chain ends up donating electrons to an anode. In FIG. 2c, Photosynthetic whole organisms (PhO) donate electrons to an anode, which in this case is a Carbon Felt electrode, via a transmembrane protein (TMP). This mediator-less electron transport is based on an intimate contact between cell and electrode.

Chamber 2 contains a catalytic cathode to reduce the hydrogen ions to hydrogen gas or alternatively to reduce oxygen and hydrogen ions to water. The panels 2d and 2e describe two alternative ways to consume the photosynthetic product (electrons, protons and oxygen) produced in chamber 1. In FIG. 2d, the cathode catalyzes the production of hydrogen gas. This reaction is not spontaneous and it requires an additional source of energy named bias potential. In FIG. 2e, the electrons are consumed in the process of reducing oxygen to water. This reaction is spontaneous and it embodies the driving force of all the system.

In FIG. 3 the effect of supplying the bias potential (current) at different voltages on hydrogen production in chamber 2 is shown in a graphical form. Significant amounts of hydrogen are produced at voltages above 860 mV.

In FIG. 4 the effect of the bias potential (current), and light, on hydrogen production from the device is shown in a graphical form. Hydrogen is produced when light is available, and when the bias potential (current) is turned on. When hydrogen is being produced, oxygen is evolved from chamber 1 at a rate of 634 nmol O₂ min⁻¹, and hydrogen is evolved from chamber 2 at a rate of 43 nmol H₂ min⁻¹, whilst electrons flow through the copper wire at 140 μC s⁻¹. The area exposed to light is 45 cm² and 25 cm² for chamber 1 and 2 respectively.

In FIG. 5(a) the effect of oxygen in chamber 2 is shown in a graphical form. Under aerobic conditions (100% O₂ equal to 260 nmol O₂ ml⁻¹), virtually no hydrogen is produced. These aerobic conditions permit the flow of spontaneous electrical current through the external circuit which is due to water production. Under strictly anaerobic conditions though, hydrogen is evolved from the platinum electrode at a rate of 67 nmol H₂ min⁻¹.

In FIG. 5(b) the rate of oxygen consumption in chamber 2 when the device is active is shown in graphical form. Under aerobic conditions (100% O₂ equal to 260 nmol O₂ ml⁻¹) oxygen is consumed at a rate of 45 nmol O₂ min⁻¹, but under anaerobic conditions, there is no oxygen available, and the rate of oxygen consumption is virtually zero.

FIG. 5(c) shows a schematic of the reactions that occur in the device when oxygen is present in chamber 2; reactions (i) and (ii) occur in chamber 1, whilst reaction (iii) occurs in chamber 2. This reaction is the driving force to support the spontaneous flow of electrical current through the external circuit. However, when the expected output is hydrogen, this reaction represents a competitive process to consume electrons and protons derived from the photosynthetic activity of chamber 1.

FIG. 5(d) shows a schematic of how hydrogen is produced from the device when chamber 2 is kept under strictly anaerobic conditions; reactions (i) and (ii) occur in chamber 1, whilst reaction (iii) occurs in chamber 2.

In FIG. 6, the effect of the individual components in a device of the invention is shown in graphical form. FIG. 6(a) shows the effect of the thylakoid concentration in chamber 1. Increasing the concentration between 0 and 15 μg chl ml⁻¹ has a significant effect on the rate of oxygenic photosynthesis, but increasing the concentration beyond this level has a small effect only; FIG. 6(b) shows the effect of altering the size of the platinum electrode. The cathode size does not change the rate of hydrogen production from chamber 2; FIG. 6(c) shows the effect of altering the surface area of the Indium Tin Oxide covered glass slide. The surface area of the anode does not influence the rate of hydrogen production; and FIG. 6(d) shows the effect of the surface area of the NAFION membrane between the two chambers. Decreasing the size of this membrane by 50% causes a 50% reduction in hydrogen production.

In FIG. 7, the effect of supplying the external energy to the device from either a power box (mains) or a photovoltaic cell is shown in graphical form. Both the power pack and photovoltaic cell are able to support equivalent rates of hydrogen production from chamber 2.

In FIG. 8, the effect of four different electron carriers on oxygenic photosynthesis in chamber 1 is shown in tabular form, along with the electrochemical properties of these four compounds. Three of these compounds are able to support oxygenic photosynthesis, and can be used as electron carriers in chamber 1.

FIG. 9 shows a comparison between hydrogen production rates from the device when ferric cyanide or dichlorophenolindophenol (DCPIP) is used as the electron carrier in chamber 1. FIG. 9(a) shows in graphical form, that when DCPIP is used as the external electron carrier the device requires a smaller bias potential (current) since the standard electrode potential of DCPIP is lower than Fe(CN)₆. FIG. 9(b) shows in graphical forms that there is no significant difference in the rate of hydrogen evolution when DCPIP or Fe(CN)₆ are used as the electron carrier, despite the fact that a smaller bias potential (current) is used when DCPIP is the electron carrier.

FIG. 10 shows a comparison between electron current production in SBD whole cell (SBD-wc) when the photosynthetic organism is floating in the chamber or is attached to the cathode.

FIG. 10a shows in graphical form that when the light is turned on and Fe(CN)₆ is used as exogenous electron carrier the SBD-wc generates ca. 350 nA cm⁻² over ca. 1100 seconds. FIG. 10b shows in graphical form that when the light is turned on the ml-SBD-wc generates ca. 7000 nA cm⁻² is generated over ca. 10000 seconds. The direct electron transport via physical contact TMP-cathode act for a certain advance in term of device performances.

### EXAMPLE 1

### Construction of Device, Hydrogen Production by Biological Method

Under conditions of sulphur deprivation the yield of hydrogen from C. reinhardtii cultures is low, because the photosynthetic electron transport chain is working under sub-optimal conditions. To alleviate this problem we developed a semi-biological device (SBD) in which the processes of photosynthesis and hydrogen production are physically separated (FIG. 1a).

### EXAMPLE 2

### Construction of Device, Electrical Current Production by SBD

When thylakoid membranes are employed as photosynthetic material, the electrical current production is time-limited because the photosynthetic membranes degrade quickly under working conditions. To address this problem we developed a semi-biological device (SBD-whole-cell) in which the photosynthetic material is a prokaryotic or eukaryotic autotrophic whole cell.

### Materials and Methods

### Construction of the Semi-Biological Device (SBD) for Hydrogen Production.

Studies were conducted in a plastic vessel, which was separated into two chambers by a NAFION cation-selective membrane. The plastic vessel was divided so that chamber 1 could hold a 200 ml solution, whilst chamber 2 could hold 60 ml. The anode in chamber 1 was an Indium Tin Oxide (ITO) coated glass electrode, whilst the cathode in chamber 2 was a platinum electrode. Anaerobic conditions in chamber 2 were created by flushing the chamber with nitrogen gas, or by chemically reducing the oxygen with sodium dithionite. The two electrodes were connected via an external electrical connection so that the potential of the cathode (in chamber 2) could be maintained at −430 mV against an Ag/AgCl reference electrode with either a power pack, or a photovoltaic (PV) cell placed underneath chamber 1 (16 cm2 PV panel). The solutions contained in both chambers were stirred with a magnetic stirring bar at 100 rpm. A tungsten bulb was used as a light source. The light was filtered through a 4 cm deep glass container filled with water, to remove ultraviolet radiation and excess heat; this resulted in a final photon flux density of 60 uE m² s⁻¹ at the surface of chamber 1. All experiments were carried out at 25° C.

Construction of the semi-biological device (SBD) for electrical current production. Studies were conducted in a plastic vessel, which was separated into two chambers by a NAFION cation-selective membrane. The plastic vessel was divided so that chamber 1 could hold a 100 μl solution, and chamber 2 could also hold 100 μl. The anode in chamber 1 was an electro conductive material (Indium Tin Oxide or Carbon Felt Electrode) coated glass electrode, whilst the cathode in chamber 2 was a platinum electrode. The two electrodes were connected via an external electrical connection, a tungsten bulb was used as a light source. The light was filtered through a 4 cm deep glass container filled with water, to remove ultraviolet radiation and excess heat; this resulted in a final photon flux density of 60 uE m² s⁻¹ at the surface of chamber 1. All experiments were carried out at 25° C.

Preparation of photosynthetic membranes. Thylakoids from Spinacia oleracea were purified as previously described. The extract was resuspended and stored in a buffer containing 200 mM sucrose, 20 mM Tricine-NaOH pH 7.5, 3 mM MgCl2 and 10 mM KCl. The chlorophyll concentration in the thylakoid preparation was determined after extraction in an 80% acetone/water solution using the extinction coefficient as described (MacKinney, 1941). The thylakoid membranes were diluted to a working concentration in running buffer (10 mM KCl, 8 mM tricine pH 7.7, 1 mM MgCl₂ and 50 μM Fe(CN)₆³⁻), before being used in chamber 1 of the SBD.

Preparation of Photosynthetic Cells. Cyanobacteria or Unicellular Algae were Grown under continuous light conditions in a medium without any organic carbon source. The chlorophyll concentration in the cells was determined after extraction in an 80% acetone/water solution using the extinction coefficient as described (MacKinney, 1941). The cells were diluted to a working concentration in running buffer (10 mM KCl, 8 mM tricine pH 7.7, 1 mM MgCl₂ and 50 μM Fe(CN)₆³⁻), before being used in chamber 1 of the SBD.

Preparation of porous anode for mediator less SBD. Cyanobacteria or Unicellular algae were grown under continuous light conditions in a medium without any organic carbon source in the presence of carbon felt electrode as anode. The chlorophyll concentration in the cells was determined after extraction in an 80% acetone/water solution using the extinction coefficient as described (MacKinney, 1941).

Analytical techniques. The current and voltage in the SBD was measured with a precision potentiostat. The red-ox state of the external electron carrier in chamber 1 was assayed spectrophotometrically; a 1 ml sample from chamber 1 was removed, centrifuged to pellet the thylakoid membranes, and the supernatant analyzed at 420 nm (for Fe(CN)₆³⁻) or 620 nm (for DCPIP). The oxygen content of the solutions in chambers 1 and 2 was assayed with a Clark electrode consisting of a silver anode and a platinum cathode in contact with the electrolyte solution. The Clark electrode was held at a constant polarising voltage of 600 mV against Ag/AgCl. Hydrogen was also measured using this amperometric (or polarographic) method. The hydrogen probe was made by modifying the Clark electrode; the platinum cathode was treated with an electrolyte containing chloroplatinic acid, whilst the silver anode was treated with an electrolyte comprising of potassium chloride. The platinized electrode was held under a constant polarizing voltage at −650 mV against Ag/AgCl.

The device is composed of two chambers separated by a NAFION™ membrane. NAFION™ allows hydrogen ions to freely pass between the chambers, but prevents the passage of all of the other components, including oxygen. Photosynthetic material in chamber 1 is used as a source of hydrogen ions and electrons. When an electron carrier is required, electrons are captured from the reducing end of photosystem I (PSI) by a soluble electron carrier. The electron carrier transports the reducing equivalents to an electrode, which then allows the electrons to flow to a thin platinum electrode placed in chamber 2. When the electron carrier is not required, electrons flow directly through transmembrane proteins to the anode. The platinum cathode catalyses the production of hydrogen, by combining hydrogen ions with electrons under anaerobic conditions. Since the terminal iron-sulphur acceptors of PSI have a red-ox midpoint of approximately −480 mV, and the midpoint of the potential of the 2H⁺/H₂ red-ox couple, at pH 7, is −420 mV at pH 7, the device is theoretically able to drive hydrogen production at the platinum cathode at the expense of light energy only. Under aerobic conditions, the platinum cathode catalyses the production of water. This spontaneous reaction is the driving force supporting the electrical current passing through the external circuit.

### EXAMPLE 3

### Operation of Device for Hydrogen Production when Thylakoid Membranes are Employed as Photosynthetic Material and a Redox Carrier is Required to Ship Electrons

To prove the feasibility of the SBD to produce hydrogen when the photosynthetic material consists of thylakoid membranes and the electrons are shipped by a soluble electron carrier, we constructed a prototype device (FIG. 1). In chamber 1 thylakoid membranes, purified from Spinacia oleracea, used as the photosynthetic material, whilst Indium Tin Oxide (ITO) coated glass was used as the electrode (FIG. 1). For the initial experiments Fe(CN)₆ was chosen as the electron carrier, since the reduction and oxidation of this compound can be measured spectrophotometrically at 420 nm. The electrode potential of the red-ox couple Fe(CN)6³⁻/Fe(CN)₆⁴⁻, at pH 7 is +420 mV, which means that with this electron carrier, the SBD is not able to produce hydrogen without an additional input of energy, because the red-ox midpoint of Fe(CN)₆³⁻/Fe(CN)₆⁴⁻ is 840 mV more positive than that of 2H⁺/H₂, making the reaction thermodynamically unfavourable. In order to drive hydrogen production from the prototype we supplied additional energy from either a power pack, or a photovoltaic (PV) cell placed underneath chamber 1. This extra input of energy was termed the “bias potential” (current). Using a power pack to supply the bias potential (current), we were able to show that the SBD does not produce significant quantities of hydrogen with Fe(CN)₆ if the additional energy is supplied at a voltage less than 840 mV, but once the voltage is increased to a value above this critical level, hydrogen is evolved from chamber 2 at a rate of 67 nmol H₂ min⁻¹. Increasing the voltage beyond 860 mV does not have a significant effect on the rate of hydrogen evolution (FIG. 3). If this electric potential is supplied to the device whilst chamber 1 is not active, hydrogen is not produced in chamber 2, indicating that the bias potential (current) (860 mV) is not able to supply the energy required for hydrogen production from the platinum electrode on its own.

The SBD was designed to use photosynthetic material to produce hydrogen using the energy from light only, but using Fe(CN)₆ as the electron carrier, the device requires a bias potential (current), and therefore an external source of energy. To investigate whether this external source of energy could be derived from light energy that was not captured by the thylakoid membranes, a PV cell was placed beneath the device. The SBD contains the thylakoid membranes in a chamber that covers 45 cm², and is 5 cm deep. A 16 cm² PV cell was placed under this chamber, such that the wavelengths of light that cannot be used by the photosynthetic membranes had to pass through chamber 1 in order to generate a current from the PV cell. The rate of hydrogen evolution using the PV panel was equivalent to the rate of hydrogen evolution using the external power pack, indicating that this panel is able to produce a sufficient input of energy (FIG. 7). Indeed, the 16 cm² PV panel produced 650 μC s⁻¹, indicating that a significantly smaller panel could be used to supply the energy that is required to drive the reaction, since an electron flow of only 140 μC s⁻¹ is required in the current device (FIG. 4).

In order to characterize the individual components of the SBD prototype, we placed a switch in the external electrical circuit (FIG. 1), which allowed the bias potential (current) derived from the PV cell to be either off or on. In the dark, the thylakoids in chamber 1 are not active; there is no significant evolution of oxygen, no significant reduction of Fe(CN)₆³⁻, nor any significant flow of current through the external circuit (FIG. 4). However, in the light, oxygen is evolved from the thylakoid membranes, and Fe(CN)₆³⁻ is reduced, but without a bias current, there is no significant flow of electrons, and no hydrogen evolution, since the reaction is thermodynamically unfavourable. As soon as the bias current is applied to the system (FIG. 4; on), electrons flow through the external circuit at 140 μC s⁻¹, and hydrogen is released from the platinum electrode at a rate of 43 nmol H₂ min⁻¹. The rate of net Fe(CN)₆³⁻ reduction is decreased in the presence of a bias potential (current) by 120 nmol min⁻¹ because the pool of Fe(CN)₆⁴⁻ is being re-oxidized at the ITO coated glass slide as the electrons are used for hydrogen production. In theory, the rate of Fe(CN)₆⁴⁻ oxidation should be twice the rate of H₂ evolution, since two electrons are required to produce H₂ from 2H⁴⁺. However in this prototype, the rate of the oxidation reaction is almost three times the rate of H₂ evolution, suggesting that not all of the electrons are being used for H₂ production (see oxygen effect (FIG. 5)). Once the pool of Fe(CN)₆³⁻ has been completely reduced to Fe(CN)₆⁴⁻ the rate of oxygen evolution decreases, since the photosynthetic activity of the thylakoid membranes is limited by the concentration of the oxidized external electron acceptor which removes electrons from the terminal PSI Fe—S clusters. However, the rate of hydrogen evolution is not affected by this reduction in photosynthetic rate (FIG. 4), since there is still a large pool of Fe(CN)₆⁴⁻ which can be re-oxidized at the ITO electrode.

Whilst the SBD overcomes the problems associated with oxygen inhibition of hydrogen production from photosynthetic microorganisms, hydrogen production from this device is actually inhibited by the presence of oxygen in chamber 2 (FIG. 5). When chamber 2 is aerobic, Fe(CN)₆³⁻ is reduced in chamber 1, but hydrogen is not produced in chamber 2. Removing approximately 95% of the oxygen from chamber 2 by bubbling argon gas through the chamber slightly increases the rate of hydrogen production. However, removing almost all of the oxygen (99.5%) from this chamber by adding dithionite to the solution causes a dramatic increase in the rate of hydrogen evolution (FIG. 5a). In the presence of oxygen, the platinum cathode preferentially catalyses the formation of water from oxygen and hydrogen ions (FIGS. 5b and 5c). However, under anaerobic conditions, when there is no oxygen available, the electrode catalyses the formation of hydrogen gas (FIG. 4d). This competitive reaction with oxygen, and the requirement for absolute anaerobic conditions in chamber 2, explains why the oxidation of Fe(CN)₆⁴⁻ at the anode was not stoichiometric with the production of hydrogen at the cathode in our initial experiments (FIG. 4).

Each individual component of the SBD has the potential to influence the rate of hydrogen evolution. To determine the optimal conditions, we sequentially altered the abundance of the individual components (FIG. 6). Changing the concentration of thylakoid membranes between 0 and 15 μg chl ml⁻¹ has a dramatic effect on the rate of oxygen evolution, but increasing the concentration of thylakoids beyond this level has no significant effect (FIG. 6a), indicating that the thylakoid membranes are able to capture almost all of the photosynthetically available radiation (PAR) at a concentration of 15 μg chl ml⁻¹. Clearly the concentration of thylakoid membranes required to capture the PAR is dependent upon the intensity of the light and the depth of chamber 1; in these experiments chamber 1 was maintained at a constant depth of 5 cm, and the light photon flux density was 60 μE m⁻² sec⁻¹.

Changing the surface area of the platinum cathode in chamber 2 from 1 cm² to 10 cm² does not influence the rate of hydrogen production under these conditions (FIG. 6b), nor does changing the surface area of the ITO covered glass from a surface area of 10 cm² to 40 cm² (FIG. 6c). However, we noticed that the performance of the device deteriorates over time in the presence of thylakoid membranes. This deterioration in performance is due to an interaction of the thylakoid membranes with the ITO surface. To prevent this deterioration, the ITO glass slides were sealed inside dialysis tubing, before being submerged in chamber 1, to stop the thylakoids physically interacting with the ITO.

The surface area of the NAFION membrane, which separates chamber 1 from chamber 2, has a significant effect on the rate of hydrogen evolution (FIG. 6d). Reducing the size of the membrane from 21 cm² to 10.5 cm² reduces hydrogen evolution by almost 50%, demonstrating that the transfer of hydrogen ions through this membrane is an important factor that influences the performance of the device under these conditions. Since the surface area of this membrane could not be made bigger that 21 cm² in this prototype, it seems likely that its surface area is the rate limiting factor for hydrogen production in all of our experiments, and explains why the rate of Fe(CN)₆³⁻ reduction is more rapid than the rate of hydrogen production (FIG. 4).

The SBD device described in this study requires an external input of energy to drive H₂ production due to red-ox potential of the electron carrier, Fe(CN)₆³⁻. We have demonstrated that the device can produce hydrogen using light energy only, if a PV cell is used to capture the wavelengths of light that are not absorbed by the photosynthetic material. An alternative electron acceptor, with a different electrode potential, could potentially minimize, or remove, the requirement for a bias current. A wide range of molecules, such as methylviologen (MV), Diaminodurone (DAD), Dicholorphenylindophenol (DCPIP) and Thymoquinone (DBMIB) are known to be active as exogenous photosynthetic electron carriers. Viologens, such as MV, appear to be promising compounds, since their electrode potential is approximately −440 mV, which would theoretically remove the requirement for a bias current. However, under aerobic conditions electron donors with an electrode potential of less than 150 mV, such as MV, donate electrons to oxygen, producing superoxide, which quickly forms H₂O₂; this reaction competes with electron donation to the ITO electrode, suppressing H₂ evolution in chamber 2. Both DCPIP and DAD are able to support oxygenic photosynthesis, and indeed they support higher rates than Fe(CN)₆³⁻ (FIGS. 8 and 9). However these compounds can accept electrons from both PSII and PSI and, in the reduced form, they can also donate electrons to PSI. Thus, the stoichiometric calculation of electron fluxes in the SBD device is hampered when such compounds are used. Nevertheless, we have run comparative experiments using DCPIP as an acceptor (which has an electrode potential of 290 mV at pH 7, showing that the SBD is functional when different electron acceptors are used (FIGS. 8 and 9). As expected from the thermodynamic properties of DCPIP, the bias current needed for hydrogen production using this electron carrier is reduced to 710 mV.

### EXAMPLE 4

### Operation of Device for Electrical Current Production when Whole Photosynthetic Cells are Employed as Photosynthetic Material and a Redox Carrier is Required to Ship Electrons

To prove the feasibility of the SBD to produce electrical current when the photosynthetic material consists of whole cells and the electrons are shipped by soluble electron carrier, we use the same prototype device (FIG. 1) that we have mentioned in Example 2. In chamber 1 the whole cells were used as the photosynthetic material, whilst Indium Tin Oxide (ITO) coated glass was used as the electrode (FIG. 1). For the initial experiments Fe(CN)₆³⁻ was chosen as the electron carrier, since the reduction and oxidation of this compound can be measured spectrophotometrically at 420 nm. The electrode potential of the red-ox couple Fe(CN)₆³⁻/Fe(CN)₆⁴⁻, at pH 7 is +420 mV, which means that with this electron carrier, the SBD is able to produce water in the cathodic chamber without an additional input of energy, because the red-ox midpoint of Fe(CN)₆³⁻/Fe(CN)₆⁴⁻ is 430 mV more negative than that of 2H⁺,2e⁻/H₂O, making the reaction thermodynamically favourable. The difference between the red-ox potential of this two couples (Fe(CN)₆³⁻/Fe(CN)₆⁴⁻ and 2H⁺,2e⁻/H₂O) represents the open circuit potential of the device.

In order to prove the production of electrical current when the photosynthetic material consists of whole cells and the electrons are shipped by soluble electron carrier, we placed a switch in the external electrical circuit (FIG. 1), which allowed us to bypass the bias potential derived from the power pack based on the branch B. In the dark, the whole cells in chamber 1 are not photosynthetically active and consequently any significant flow of current through the external circuit (FIGS. 1 and 4). However, in the light, electrons flow through the external circuit at 350 nC s-1 cm-2, and water is released from the cathode electrode. In theory, the rate of Fe(CN)₆⁴⁻ oxidation should be 4 times the rate of H₂O evolution, since 4 electrons are required to produce H₂O from 2 electron, 2H⁺ and ½ dioxygen.

Whilst the SBD run by whole photosynthetic cells overcomes the problems associated with the short lifespan of thylakoid membranes, the electrical current production from this device is actually limited by the availability of electrons. When the photosynthetic material performs oxygenic photosynthesis, the electrons obtained by water photolysis are kept at chloroplast level, surrounded by phospholipidic membranes and virtually inaccessible by water-soluble electron carriers. Through the exploitation of the electrogenic activity of endogenous transmembrane proteins, a portion of these electrons can be donated to an electron carrier resulting in electrical current production. In the presence of oxygen, the platinum cathode preferentially catalyses the formation of water, combining oxygen, electrons and hydrogen ions. This spontaneous reaction is the driving force for all the process and keeps it thermodynamically favourable.

The SBD device described in this study does not require an external input of energy to drive electrical current production. We have demonstrated that the device can produce a flux of electrons through the external circuit using light energy only. An alternative electron acceptor, with a different electrode potential, could potentially maximize the open circuit potential and dramatically increase the output of electrical current of our SBD.

### EXAMPLE 5

### Operation of Device for Electrical Current Production when Whole Photosynthetic Cells are Employed as Photosynthetic Material and a Redox Carrier is not Required

We have proven with our prototype device (FIG. 1) the feasibility of the SBD to produce electrical current when the photosynthetic material consists of whole cells and the electrons are directly shipped to the anode without any soluble electron carrier. In chamber 1 whole cells where used as photosynthetic material, whilst Carbon Felt Electrode was used as the anode (FIG. 1). For the initial experiments the cells were grown on the electrode leading to the formation of photosynthetic biofilm on the electrode surface. The electrode potential of the transmembrane protein is relatively negative, which means that with this electron donor, the SBD is able to produce water in the cathodic chamber without an additional input of energy, because the red-ox midpoint of Fe(CN)₆³⁻/Fe(CN)₆⁴⁻ is 430 mV more negative than that of 2H⁺,2e⁻/H₂O, making the reaction thermodynamically favourable. The difference between the red-ox potential of the transmembrane proteins and the oxygen reduction (2H⁺,2e⁻/H₂O) represents the open circuit potential of the device.

In order to prove the production of electrical current when the photosynthetic material consists of whole cells and the electron carrier is not required, we placed a switch in the external electrical circuit (FIG. 1), which allowed us to bypass the bias potential derived from the power pack based on the branch B. In the dark, the whole cells in chamber 1 are not photosynthetically active and consequently any significant flow of current through the external circuit is not expected (FIGS. 1 and 4), however, the metabolic activity of the cells supports a residual flux of electrons. In the light, electrons flow through the external circuit at ca. 7000 nC s⁻¹ cm⁻², and water is released from the cathode electrode.

Whilst the mediator-less SBD driven by whole photosynthetic cells overcomes the problems associated with the short life time of thylakoid membranes and enhances the current peak, the electrical current production from this device is limited by the availability of electrons. When the photosynthetic material performs oxygenic photosynthesis, the electrons obtained by water photolysis are kept in the chloroplast. Through the exploitation of the electrogenic activity of endogenous transmembrane proteins and the direct contact of these proteins with the anode, a portion of the electrons produced by oxygenic photosynthetic activity can be passed to the anode resulting in electrical current production. In the presence of oxygen, the platinum cathode preferentially catalyses the formation of water combining oxygen, electrons and hydrogen ions. This spontaneous reaction is the driving force and it keeps the process thermodynamically favourable.

The SBD device described in this study does not require an external input of energy to drive electrical current production. We have demonstrated that the device can produce a flux of electrons through the external circuit using light energy only. Enhancing the activity of transmembrane proteins, increasing their number, engineering their molecular structure or developing a new strategy of direct electron transport (using conductive “pili”, for example) could potentially maximize the open circuit potential and dramatically increase the performance of our electrochemical SBD.

In conclusion, we have developed a novel device in which photosynthetic material can be used to produce hydrogen gas and electrical current in the presence of oxygen. This method overcomes many of the problems associated with biological hydrogen production and biological electrical current production.

In particular:
a) hydrogen production from the SBD is not inhibited by molecular oxygen, and oxygen and hydrogen are produced in separate compartments, which prevents the two gases mixing into an explosive cocktail. The isolated thylakoid membranes used in this prototype deteriorate over time, but we have shown that it is possible to use intact cells (SBD-whole cell and mediator-less SBD whole cell).
b) It is important to consider that a limiting for factor hydrogen production by the SBD is the need for an additional energy source; this requirement is related to the red-ox potential of the electron carrier. This additional energy requirement can be removed by using electron carriers with a more negative red-ox potential. However, electron carriers with such negative electrode potentials react with oxygen to produce superoxide, so care must be taken in order to reduce these undesirable reactions.
c) Electrical current production by SBD-whole cell employing an exogenous electron carrier overcomes the limited life time of previous technology based on thylakoid membranes even though it was thought difficult to access the electrons that are available inside intact cells.
d) Electrical current production from mediator-less SBD-whole cell employing biofilms of intact photosynthetic organisms on the anodic electrode enhances the rate of electron transport (cell→anode). Even though it is still difficult to get access to all photosynthetic electrons that are available inside intact cells, the rapid progress in technology associated with photo-electrochemical cells and mediator-less microbial fuel cells is likely to allow intact cells to be used with a high degree of efficiency.

The current quantum efficiency of the SBDs is between 1 and 3%, which is significantly higher than that produced from current biological methods using photosynthetic organisms. The SBD exhibits many unique and attractive attributes in the framework of renewable energy sources; hydrogen is produced from sunlight that is freely available, the core biological material is self-assembling, hydrogen is produced in a separate chamber to oxygen and is therefore virtually pure, and greenhouse gases are not generated in the production process. SBD-whole cells show an enhanced life time and the development of mediator-less SBD is likely to allow intact cells to be used with a high degree of efficiency.

The economic benefits of bio-hydrogen and bio current production are unavoidably linked with the development of new effective technologies and their subsequent improvement. The SBDs represents an important, novel technology, which has the potential to be developed into an economically viable hydrogen production system.

1. A device for the generation of hydrogen by oxygenic photosynthesis comprising a first chamber and a second chamber, the first chamber having two arrangements in which either (1) the first chamber has an anode in contact with an aqueous solution comprising a photosynthetic organism and optionally an electron acceptor molecule, or a photosynthetic part of said photosynthetic organism and an electron acceptor molecule, an inlet and an outlet, or (2) the first chamber has an anode in direct contact with a photosynthetic organism, the second chamber having a cathode in contact with an aqueous solution of an electrolyte under anaerobic conditions, an outlet for the release of hydrogen, wherein the anode and the cathode are connected by a switched electric circuit having an external power source to supply a bias potential and wherein the second chamber is separated from the first chamber by a proton selective membrane, in which hydrogen is produced on application of light to the first chamber by reduction of hydrogen ions to hydrogen gas.

2. A device for the generation of electric current by oxygenic photosynthesis comprising a first chamber and a second chamber, the first chamber having two arrangements in which either (1) the first chamber has an anode in contact with an aqueous solution comprising a photosynthetic organism and optionally an electron acceptor molecule or a photosynthetic part of said photosynthetic organism and an electron acceptor molecule, an inlet and an outlet, or (2) the first chamber has an anode in direct contact with a photosynthetic organism, the second chamber having a cathode in contact with an aqueous solution of an electrolyte under aerobic conditions, an outlet, wherein the anode and the cathode are connected by a switched electric circuit and wherein the second chamber is separated from the first chamber by a proton selective membrane, in which electric current is generated on application of light to the first chamber by reduction of oxygen to water at the cathode.

3. A device as claimed in claim 1, in which the first and second chambers are arranged such that the second chamber is contained within the first chamber.

4. A device as claimed in claim 1, in which the first and second chambers are constructed as adjacent chambers in which the connecting surface between the adjacent chambers is the proton selective membrane.

5. A device as claimed in claim 1, in which the photosynthetic organism or part thereof is a thylakoid or a thylakoid membrane, photosynthetic bacteria, eukaryotic algae, plant or plant tissue.

6. A device as claimed in claim 5, in which the thylakoid membrane is prepared from plant tissue from a terrestrial plant or an aquatic plant.

7. A device as claimed in claim 6, in which the plant tissue is from spinach, lettuce, beet, cereals, or grass.

8. A device as claimed in claim 6, in which the plant tissue is from Posidoniaceae, Zosteraceae, Zostera, Heterozostera, Phyllospadix, Enhalus, Halophila, Thalassia, Amphibolis, Cymodocea, Halodule, Syringodium, or Thalassodendron.

9. A device as claimed in claim 5, in which the photosynthetic bacteria are cyanobacteria.

10. A device as claimed in claim 5, in which the cyanobacteria are Anabaena, Crocosphaeri, Phormidium, Gloeobacter, Nostoc punctiforme, Nostoc sp., Prochlorococcus marinus, Synechococcus elongatus, Synechococcus sp, Thermosynechococcus elongatus, or Trichodesmium erythraeum.

11. A device as claimed in claim 5, in which the eukaryotic algae are Antithamnion, Ascophyllum, Atractophora, Audouinella, Botryococcus, Charales, Chlamydomonas, Chlorella, Chlorogonium, Chondrus, Cladophora, Codium, Coleochaete, Corallina, Cryptomonas, Cyanidioschyzon, Cyanidium, Dasya, Desmids, Dunaliella, Dysmorphococcus, Enteromorpha, Euglena, Falosphaera, Fucus, Haematococcus, Isochrysis, Laminaria, Lemanea, Mougeotia, Nannochloris, Nannochloropsis, Neochloris, Pelvetia, Phacotus, Phaeodactylum, Platymonas Pleurochrysis, Polytoma, Polytomella, Porphyridium, Prymnesium, Pyramimonas, Scenedesmus, Spirogyra, Spirulina, Spyridia, Tetraselmis, Tetraspora, Thalassiosira, Ulva, Volvox, or Zygnema.

12. A method for the generation of hydrogen from a device according to claim 1 comprising:
providing a photosynthetic organism or part thereof to the first chamber of the device;
providing an aqueous solution of an electrolyte in the second chamber of the device under anaerobic conditions;
applying light to the first chamber;
operating the switch to connect the anode to the cathode; and
introducing a source of additional electron motive force from an external power source,
wherein hydrogen is generated at the cathode by reduction of hydrogen ions to hydrogen gas.

13. A method for the generation of an electrical current from a device according to claim 2, comprising:
providing a photosynthetic organism or part thereof to the first chamber of the device;
providing an aqueous solution of an electrolyte in the second chamber of the device under aerobic conditions;
applying light to the first chamber;
operating the switch to connect the anode to the cathode; and
wherein electrical current is generated at the cathode by reduction of oxygen to water.

14. A device as claimed in claim 2, in which the first and second chambers are arranged such that the second chamber is contained within the first chamber.

15. A device as claimed in claim 2, in which the first and second chambers are constructed as adjacent chambers in which the connecting surface between the adjacent chambers is the proton selective membrane.

16. A device as claimed in claim 2, in which the photosynthetic organism or part thereof is a thylakoid or a thylakoid membrane, photosynthetic bacteria, eukaryotic algae, plant or plant tissue.

17. A device as claimed in claim 16, in which the thylakoid membrane is prepared from plant tissue from a terrestrial plant or an aquatic plant.

18. A device as claimed in claim 17, in which the plant tissue is from spinach, lettuce, beet, cereals, or grass.

19. A device as claimed in claim 17, in which the plant tissue is from Posidoniaceae, Zosteraceae, Zostera, Heterozostera, Phyllospadix, Enhalus, Halophila, Thalassia, Amphibolis, Cymodocea, Halodule, Syringodium, or Thalassodendron.

20. A device as claimed in claim 16, in which the photosynthetic bacteria are cyanobacteria.

21. A device as claimed in claim 16, in which the cyanobacteria are Anabaena, Crocosphaeri, Phormidium, Gloeobacter, Nostoc punctiforme, Nostoc sp., Prochlorococcus marinus, Synechococcus elongatus, Synechococcus sp, Thermosynechococcus elongatus, or Trichodesmium erythraeum.

22. A device as claimed in claim 16, in which the eukaryotic algae are Antithamnion, Ascophyllum, Atractophora, Audouinella, Botryococcus, Charales, Chlamydomonas, Chlorella, Chlorogonium, Chondrus, Cladophora, Codium, Coleochaete, Corallina, Cryptomonas, Cyanidioschyzon, Cyanidium, Dasya, Desmids, Dunaliella, Dysmorphococcus, Enteromorpha, Euglena, Falosphaera, Fucus, Haematococcus, Isochrysis, Laminaria, Lemanea, Mougeotia, Nannochloris, Nannochloropsis, Neochloris, Pelvetia, Phacotus, Phaeodactylum, Platymonas Pleurochrysis, Polytoma, Polytomella, Porphyridium, Prymnesium, Pyramimonas, Scenedesmus, Spirogyra, Spirulina, Spyridia, Tetraselmis, Tetraspora, Thalassiosira, Ulva, Volvox, or Zygnema.
